(19) United States
(12) Patent Application Publication
(10) Pub. No.: US 2010/0029895 A1
(43) Pub. Date: Feb. 4, 2010

(54) Fluorinated Diamine and Polymer Formed Therefrom

(75) Inventors: Satoru Narizuka, Saitama (JP); Yuji Hagiwara, Saitama (JP); Kazuhiro Yamanaka, Tokyo (JP)

Correspondence Address: CROWELL & MORING LLP;INTELLECTUAL PROPERTY GROUP, P.O. BOX 14300, WASHINGTON, DC, 20044-4300, US

(73) Assignee: Central Glass Company, Limited, Ube-shi (JP)

(21) Appl. No.: 12/519,428

(22) PCT Filed: Nov. 13, 2007
(86) PCT No.: PCT/JP2007/071980

§ 371 Date: Jun. 16, 2009

(30) Foreign Application Priority Data
Dec. 19, 2006 (JP) 2006-341834

(51) Int. Cl. C08G 69/28; C07C 211/56; C08G 69/26

(52) U.S. Cl. 528/344; 564/427; 564/428; 528/353

(57) ABSTRACT

There is provided a fluorine-containing diamine represented by formula (1).

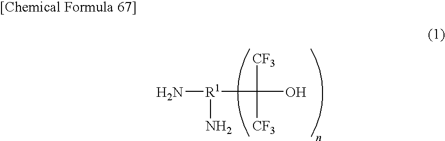

In this formula, R¹ represents a condensed polycyclic type aromatic hydrocarbon group, and at least one —C(CF₃)₂OH group and at least one —NH₂ group are in a relation such that they are attached to adjacent carbons of carbon atoms constituting the condensed polycyclic type aromatic hydrocarbon group. Polymer compounds derived from this fluorine-containing diamine have superior low dielectric property and low water-absorbing property, and, in addition to that, shows low thermal expansion property and high glass transition temperature.

### TECHNICAL FIELD

The present invention relates to a novel, fluorine-containing, rigid diamine and novel polymer compounds using the same

### BACKGROUND OF THE INVENTION

Polyamide and polyimide have been developed as representatives of organic polymers having high-degree heat resistance. They form a large market in electronic device field, engineering plastic field such as automotive and aerospace uses, fuel cell field, medical material field, optical material field, etc. At their center, many various polymers are put into practical use, such as polyamide represented by nylon, KEVLAR, etc.; polyamide acid and polyimide, which can be representative heat-resistant polymers; polyamide imide, which is a composite of them; and polybenzoxazole, polybenzthiazole, polybenzimidazole, etc. In particular, polyimide is again recently attracting attention as a material that is resistant to a lead-free solder step.

Polymerization of many of these heat-resistant polymers is conducted by successively generating a reaction, such as polyaddition or polycondensation, using plural types of monomers having a bifunctional or trifunctional reactive group in the molecule.

Regarding combinations of monomers in polymerization, there are known, in the case of polyamide, a method in which a diamine-type monomer is condensed with dicarboxylic acid or a dicarboxylic acid derivative, such as acid chloride or ester, and, in the case of polyamide acid or polyimide, a method by a polyaddition of diamine and acid dianhydride. As diamines that are generally used, aliphatic diamines, alicyclic diamines and aromatic diamines have been reported. From the viewpoint of polymerizability and heat resistance, however, there is preferably used an aniline-series monomer that has a supporting skeleton of a benzene single ring, biphenyl-type or polycyclic structure, in which a plurality of benzene rings are directly or indirectly bonded together, and that contains a plurality of amines in the molecule On the other hand, in the case of polybenzoxazole and polybenzthiazole, there are used monomers having amine and hydroxyl group and amine and thiol group at ortho-positions of the benzene ring.

The purpose of simultaneously having an amine and another functional group in the molecule is explained, as follows. That is, there is conducted a design in which diamine is used as a polymerization site and at the same time hydroxyl group and thiol group are used as functional groups for intramolecular condensation cyclization, and in which a phenolic acid group is contained as a photosensitive functional group of these alkali-soluble groups and the like. However, there are reports of only the above-mentioned limited combinations in an attempt to contain plural types of functional groups together with diamine.

On the other hand, fluoro compounds have been developed or put into practical use in wide material fields, such as polyolefins and condensation polymers, mainly in advanced material fields, due to characteristics possessed by fluorine, such as water repellency, oil repellency, low water absorptive property, heat resistance, weather resistance, corrosion resistance, transparency, photosensitivity, low refractive index property and low dielectric property. In the condensation polymer field, an attempt to introduce fluorine into a diamine monomer has been conducted. There are reports of a diamine monomer in which hydrogen of the benzene ring has been replaced with fluorine atom or trifluoromethyl group, a diamine monomer in which a hexafluoroisopropenyl group has been introduced between two aromatic rings, and a fluorine-containing diamine monomer in which the benzene ring has been subjected to a hydrogen reduction. Furthermore, a bishydroxyamine monomer having a hexafluoroisopropenyl group as a central atomic group and aromatic hydroxyamines at its both sides has also been put into practical use. In this case, it is applied as a polybenzoxazole or hydroxy group-containing polyimide.

They are explained, for example, as fluorine-containing polybenzazoles in Non-patent Publication 1, etc. On the other hand, there have recently been conducted active researches and developments on photoresist materials, in which transparency of fluoro compounds in ultraviolet region, particularly in vacuum ultraviolet wavelength region, has been applied. It is an attempt to achieve adhesion to substrate, high glass transition point, photosensitivity due to acidity of fluorocarbinol group, alkali development property, etc., while achieving transparency at each wavelength for use by introducing fluorine. In particular, of fluorocarbinols, hexafluoroisopropyl group attracts attention due to its dissolution behavior, non-swelling property, high contrast, etc., and many researches and developments are conducted.

As assumed from photoresist development examples, hexafluoroisopropyl group, which is an acidic alcohol, has a potential for achieving a rapid, homogeneous, alkali solubility, while it maintains less swelling property. There have been, however, only a few reports of development examples of heat resistant polymers using a similar concept, that is, heat resistant polymers containing a hexafluoroisopropyl group as an acidic alcohol (Patent Publications 1, 2 and 3). Carboxylic group can be cited as a general acidic group. However, due to its high reactivity with amine, they say that it is difficult to make an amine having a carboxylic group in the same molecule exist stably.
Non-patent Publication 1: “Latest Polyimide, its basic and application” edited by Japan Polyimide Study Group, page 426, (published in 2002)
Patent Publication 1: Japanese Patent Application Publication 2006-206879
Patent Publication 2: International Application Publication 2006/041115 Pamphlet
Patent Publication 3: International Application Publication 2006/043501 Pamphlet

### SUMMARY OF THE INVENTION

The fluorine-containing heat resistant resins, which are described in Patent Publications 2 and 3, are fluorine-containing resins containing a hetero ring, in which a fluoroalkyl group has been substituted, as novel heat resistant resins that replace conventional polyimide and polybenzoxazole. The fluorine-containing resins can be synthesized at a lower temperature (250° C.) as compared with those (320-350° C.) of conventional polyimide and polybenzoxazole. To be capable of synthesis at low temperature contributes to relaxation of the residual thermal stress in uses of electronic materials such as LSI. Therefore, we can say that they are resins provided with very useful characteristics. There is a report that furthermore the resins also show lower dielectric constant and lower water-absorbing property, as compared with conventional polyimide and polybenzoxazole.

However, the fluorine-containing heat resistant resins, which are described in Patent Publications 2 and 3, had a problem that they were inferior in thermal characteristics due to the tendency to have a lower glass transition temperature and a growing thermal expansion coefficient as compared with conventional polyimide and polybenzoxazole.

That is, it was a task to find a novel fluorine-containing diamine for improving heat resistant resins described in Patent Publications 2 and 3 and by using it to provide a polymer compound having superior thermal stability (high thermal decomposition temperature, high glass transition temperature, and low thermal expansion coefficient), characteristics (water repellency, oil repellency, etc.) as fluorine-containing materials, resistances (weather resistance, corrosion resistance, etc.), other characteristics (transparency, low refractive index property, low dielectric property, etc.) and alkali solubility, photosensitivity, organic solvent solubility, etc. In particular, it was a task to provide a polymer compound having low dielectric property and low water-absorbing property, which are derived from fluorine, and, besides that, showing low thermal expansion property and high glass transition temperature.

As a result of a repeated eager study to solve the task, the present inventors have found a novel diamine compound which has two amino groups by centering a condensed polycyclic aromatic hydrocarbon and in which at least one hydrogen atom adjacent to the amino groups has been replaced with a hexafluoroisopropyl group, and novel polymer compounds obtained by using the same.

As mentioned above, although the fluorine-containing heat resistant resins described in Patent Publications 2 and 3 show characteristics such as low temperature curing property, low dielectric property and low water-absorbing property, they had a part inferior in heat resistance. Thus, in the present invention, there was found a novel diamine compound containing a condensed polycyclic hydrocarbon structure for improving heat resistance.

It was found that the diamine compound shows good polymerization characteristic and that polyamide type polymer compounds represented by formulas (10), (12), (14), (17), etc. can be produced with good yield by making a contact with the after-mentioned dicarboxylic acid derivative represented by formula (22) or (23) or tetracarboxylic acid dianhydride monomer represented by formula (24) and conducting a reaction in a predetermined temperature region.

As a result of further subjecting the polymers to dehydration and ring closure, it was found that hetero ring type polymer compounds represented by formulas (11), (13), (15), (16), (18), etc. are obtained with good yields.

It was found that, while the hetero ring type polymer compounds maintain properties of low temperature curing property, low dielectric property and low water-absorbing property at levels almost comparable to the fluorine-containing hetero ring polymers described in Patent Publications 2 and 3, they show remarkably superior heat resistant characteristics (high glass transition temperature and further low thermal expansion coefficient) as compared with these polymers.

For example, also in Comparative Examples 1 and 2 (after-mentioned) of the present specification, polymers containing trifluoromethyl groups having hetero aromatic rings having relatively rigid structures have been synthesized, but it was not possible to say that heat resistance characteristics were sufficient. Particularly as in Comparative Example 2, a biphenylene skeleton (the right-end part of formula (30)), which is expected to be higher in rigidity, has been introduced, but heat resistance characteristics have not sufficiently been improved.

In contrast with this, a polymer of the present invention shows a structure in which an aromatic ring has been incorporated into an inner skeleton of diamine in the form of condensed ring. As a result of an increased rigidity of this inner skeleton, it shows that heat resistance characteristics have been improved remarkably.

That is, the present inventors have found the after-mentioned hetero ring type polymer compounds represented by formulas (11), (13), (15), (16), (18), etc., and have found that the hetero ring type polymer compounds show superior low temperature curing property, low dielectric property and low water-absorbing property and at the same time heat resistance characteristics (high glass transition temperature and low thermal expansion property) superior to conventional ones. Furthermore, we have found polyamide type polymer compounds represented by formulas (10), (12), (14), (17), etc., which are intermediates for producing the hetero ring type polymer compounds. Furthermore, we have found novel diamine compounds for producing the polyamide type polymer compounds.

Furthermore, the inventors have found processes for producing these polymer compounds and completed the invention. That is, according to the present invention, the following novel diamine compounds, polymer compounds using the same, and processes for producing them are provided.

According to the present invention, there is provided a fluorine-containing diamine (a first diamine) represented by formula (1).

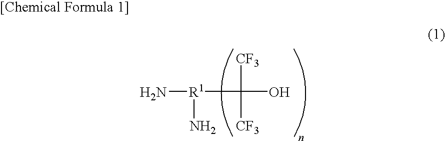

[In the formula, R¹ represents a condensed polycyclic type aromatic hydrocarbon group, and the condensed polycyclic type aromatic hydrocarbon group may contain N atom, O atom or S atom as a hetero atom and may have a functional group containing N atom, O atom or S atom as a substituent. n represents an integer of 1 or greater. However, at least one —C(CF₃)₂OH group and at least one —NH₂ group are in a relation such that they are attached to adjacent carbons of carbon atoms constituting the condensed polycyclic type aromatic hydrocarbon group.]

The first diamine may be a fluorine-containing diamine (a second diamine) represented by formula (2).

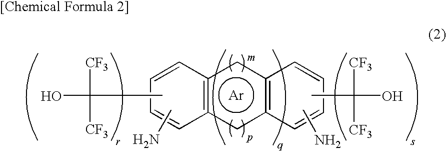

[In the formula, each of m and p is independently an integer of 0, 1 or 2, and m+p≦2. q is 0 or an integer of 1 or greater. Each of r and s is independently an integer of 0-3, and (r+s) is 1 or greater. However, at least one —C(CF₃)₂OH group and at least one —NH₂ group are in a relation such that they are attached to adjacent carbons of carbon atoms constituting the condensed polycyclic type aromatic hydrocarbon group.

Furthermore, in the formula, a part represented by the following formula

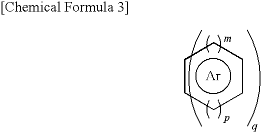

represents a monocyclic type aromatic ring or condensed polycyclic type aromatic ring, may contain N atom, O atom or S atom as a hetero atom, and may have a functional group containing N atom, O atom or S atom as a substituent.]

The second diamine may be a fluorine-containing diamine (a third diamine) represented by formula (3).

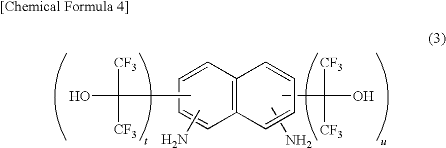

[In the formula, each of t and u is independently an integer of 0-3, and (t+u) is 1 or greater. However, at least one —C(CF₃)₂OH group and at least one —NH₂ group are in a relation such that they are attached to adjacent carbons of carbon atoms constituting the condensed polycyclic type aromatic hydrocarbon group.]

The second diamine may be a fluorine-containing diamine (a fourth diamine) represented by formula (4).

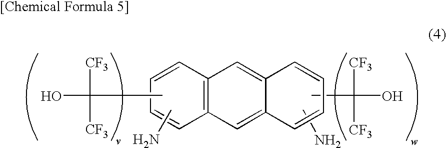

[In the formula, each of v and w is independently an integer of 0-3, and (v+w) is 1 or greater. However, at least one —C(CF₃)₂OH group and at least one —NH₂ group are in a relation such that they are attached to adjacent carbons of carbon atoms constituting the condensed polycyclic type aromatic hydrocarbon group.]

The second diamine may be a fluorine-containing diamine (a fifth diamine) represented by formula (5).

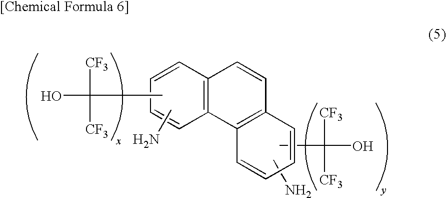

[In the formula, each of x and y is independently an integer of 0-3, and (x+y) is 1 or greater. However, at least one —C(CF₃)₂OH group and at least one —NH₂ group are in a relation such that they are attached to adjacent carbons of carbon atoms constituting the condensed polycyclic type aromatic hydrocarbon group.]

The first diamine may be a fluorine-containing diamine (a sixth diamine) represented by formula (6).

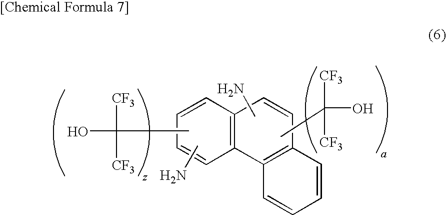

[In the formula, a is 0 or 1, z is an integer of 0-3, and (a+z) is 1 or greater. However, at least one —C(CF₃)₂OH group and at least one —NH₂ group are in a relation such that they are attached to adjacent carbons of carbon atoms constituting the condensed polycyclic type aromatic hydrocarbon group.]

The third diamine may be a fluorine-containing diamine (a seventh diamine) represented by formula (7).

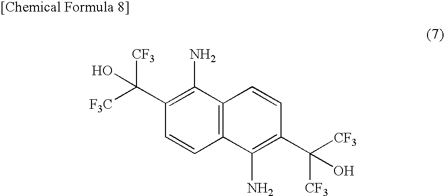

The third diamine may be a fluorine-containing diamine (an eighth diamine) represented by formula (8).

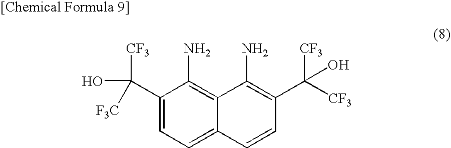

The fourth diamine may be a fluorine-containing diamine (a ninth diamine) represented by formula (9).

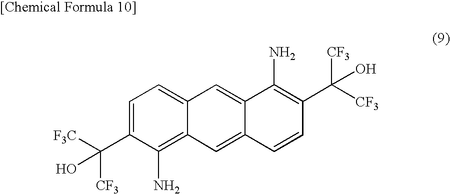

According to the present invention, there is provided a first polymer compound obtained by bringing any one of the first to ninth diamines into contact with a dicarboxylic acid derivative represented by formula (22) or (23)

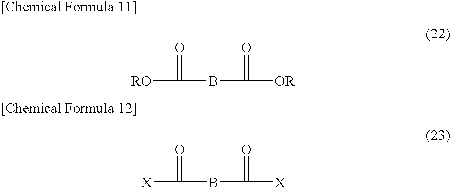

or a tetracarboxylic acid dianhydride monomer represented by formula (24)

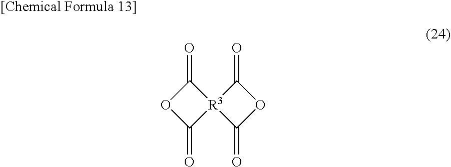

thereby conducting a reaction.

Furthermore, according to the present invention, there is provided a second polymer compound obtained by subjecting the first polymer compound to dehydration and ring closure.

Furthermore, according to the present invention, there is provided a third polymer compound at least containing a repeating unit represented by the following formula (10).

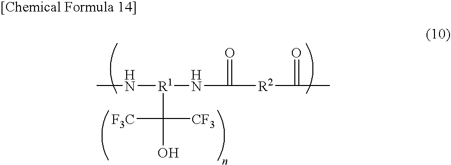

[In the formula, R¹ represents a condensed polycyclic type aromatic hydrocarbon group, and the condensed polycyclic type aromatic hydrocarbon group may contain N atom, O atom or S atom as a hetero atom and may have a functional group containing N atom, O atom or S atom as a substituent. n represents an integer of 1 or greater. R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided a fourth polymer compound at least containing a repeating unit represented by the following formula (11).

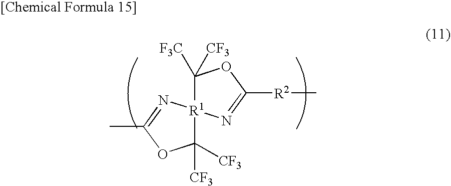

[In the formula, R¹ represents a condensed polycyclic type aromatic hydrocarbon group, and the condensed polycyclic type aromatic hydrocarbon group may contain N atom, O atom or S atom as a hetero atom and may have a functional group containing N atom, O atom or S atom as a substituent. R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided a fifth polymer compound at least containing a repeating unit represented by the following formula (12).

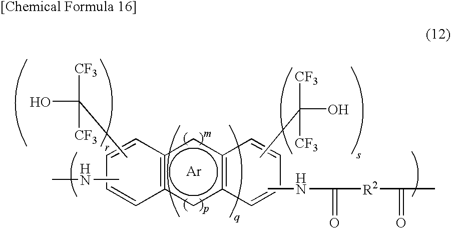

[In the formula, each of m and p is independently an integer of 0, 1 or 2, and m+p≦2. q is 0 or an integer of 1 or greater. Each of r and s is independently an integer of 0-3, and (r+s) is 1 or greater. However, at least one —C(CF₃)₂OH group and at least one —NH₂ group are in a relation such that they are attached to adjacent carbons of carbon atoms constituting the condensed polycyclic type aromatic hydrocarbon group. R² is a bivalent organic group containing at least one selected from aliphatic rings, aromatic rings and alkylene groups, and may contain fluorine, chlorine, oxygen, sulfur or nitrogen, and the hydrogens may partly be replaced with an alkyl group, fluoroalkyl group, carboxyl group, hydroxy group or cyano group.

Furthermore, in the formula, a part represented by the following formula

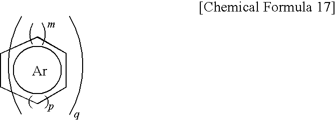

represents a monocyclic type aromatic ring or condensed polycyclic type aromatic ring, may contain N atom, O atom or S atom as a hetero atom, and may have a functional group containing N atom, O atom or S atom as a substituent.]

Furthermore, according to the present invention, there is provided a sixth polymer compound at least containing a repeating unit represented by the following formula (13).

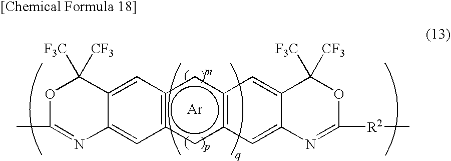

[In the formula, each of m and p is independently an integer of 0, 1 or 2, and m+p≦2. q is 0 or an integer of 1 or greater. R² is a bivalent organic group containing at least one selected from aliphatic rings, aromatic rings and alkylene groups, and may contain fluorine, chlorine, oxygen, sulfur or nitrogen, and the hydrogens may partly be replaced with an alkyl group, fluoroalkyl group, carboxyl group, hydroxy group or cyano group.

Furthermore, in the formula, a part represented by the following formula

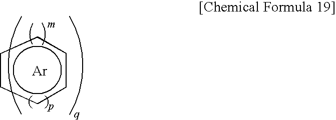

represents a monocyclic type aromatic ring or condensed polycyclic type aromatic ring, may contain N atom, O atom or S atom as a hetero atom, and may have a functional group containing N atom, O atom or S atom as a substituent.]

Furthermore, according to the present invention, there is provided a seventh polymer compound at least containing a repeating unit represented by the following formula (14).

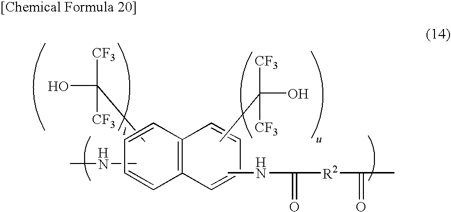

[In the formula, each of t and u is independently an integer of 0-3, and (t+u) is 1 or greater. However, at least one —C(CF₃)₂OH group and at least one —NH₂ group are in a relation such that they are attached to adjacent carbons of carbon atoms constituting the condensed polycyclic type aromatic hydrocarbon group. R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided an eighth polymer compound at least containing a repeating unit represented by the following formula (15).

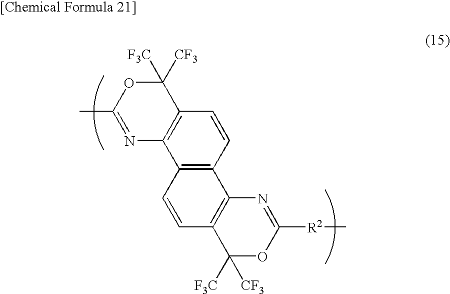

[In the formula, R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided a ninth polymer compound at least containing a repeating unit represented by the following formula (16).

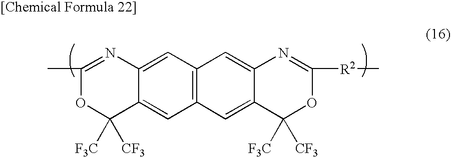

[In the formula, R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided a tenth polymer compound at least containing a repeating unit represented by the following formula (17).

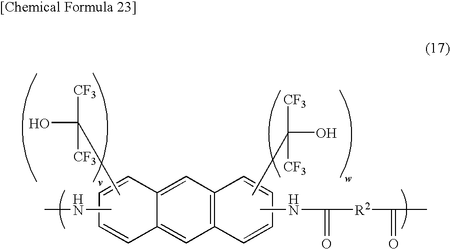

[In the formula, each of v and w is independently an integer of 0-3, and (v+w) is 1 or greater. However, at least one —C(CF₃)₂OH group and at least one —NH₂ group are in a relation such that they are attached to adjacent carbons of carbon atoms constituting the condensed polycyclic type aromatic hydrocarbon group. R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided an eleventh polymer compound at least containing a repeating unit represented by the following formula (18).

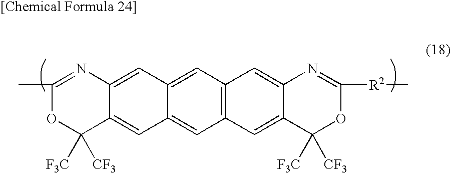

[In the formula, R² is a bivalent organic group containing at least one selected from aliphatic rings, aromatic rings and alkylene groups, and may contain oxygen, sulfur or nitrogen as a hetero atom, and the hydrogens may partly be replaced with an alkyl group, fluorine, chlorine, fluoroalkyl group, carboxyl group, hydroxy group or cyano group.]

Furthermore, according to the present invention, there is provided a process for producing the fourth polymer compound by bringing the first diamine into contact with a dicarboxylic acid derivative represented by formula (22) or (23)

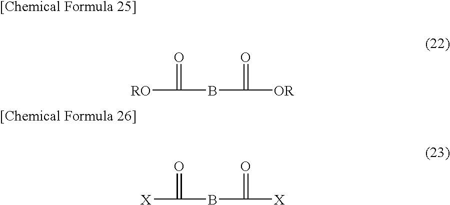

or a tetracarboxylic acid dianhydride monomer represented by formula (24)

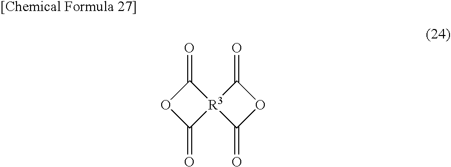

thereby conducting a reaction to produce the third polymer compound, followed by subjecting the third polymer compound to dehydration and ring closure. [In the formulas (22) and (23), each R is independently a group selected from hydrogen, C₁₋₁₀ alkyl groups and benzyl group, B is a bivalent organic group containing at least one selected from aliphatic rings, aromatic rings and alkylene groups, and may contain oxygen, sulfur or nitrogen, and the hydrogens may partly be replaced with an alkyl group, fluorine, chlorine, fluoroalkyl group, carboxyl group, hydroxy group or cyano group. X represents a halogen atom (chlorine, fluorine, bromine or iodine).

In the formula (24), R³ is a tetravalent organic group containing at least one selected from aliphatic rings, aromatic rings and alkylene groups, and may contain fluorine, chlorine, oxygen, sulfur, nitrogen, etc., and the hydrogens may partly be replaced with an alkyl group, fluoroalkyl group, carboxyl group, hydroxy group or cyano group.

### DETAILED DESCRIPTION

A polymer compound derived from a novel diamine compound which has two amino groups by centering a condensed polycyclic aromatic hydrocarbon and in which at least one hydrogen atom adjacent to the amino groups has been replaced with a hexafluoroisopropyl group has superior low dielectric property and low water-absorbing property derived from a fluorine-containing hetero ring and in addition shows low thermal expansion property and high glass transition temperature. As a result of this, it becomes possible to provide various superior materials that are balanced in a plurality of properties.

The task is solved, for example, by the sixth polymer compound (a hetero ring type high polymer compound). This polymer has a structure in which an aromatic ring in the form of condensed ring has been incorporated into an inner skeleton of diamine and, as a result of an increased rigidity of the inner skeleton, is remarkably superior in heat resistance characteristics (low thermal expansion coefficient and high glass transition temperature), while maintaining induction property and low water-absorbing property of conventional fluorine resins. Therefore, it can preferably be used for various materials.

In the following, the present invention is described in more detail.

As the condensed aromatic hydrocarbon group R¹ in the diamine compound represented by formula (1)

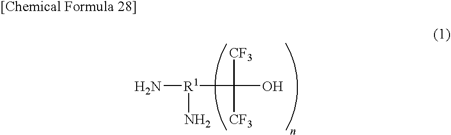

which is provided by the present invention, is specifically exemplified, it is possible to preferably cite compounds, such as pentalene, indene, naphthalene, azulene, heptalene, biphenylene, indacene, acenaphtylene, fluorene, phenalene, phenanthrene, anthracene, fluoranthene, acephenanthrylene, aceanthrylene, triphenylene, pyrene, chrysene, naphthacene, picene, pentaphene, pentacene, tetraphenylene, hexaphene, hexacene, rubicene, coronene, trinaphthylene, heptaphene, heptacene, pyranthrene, and ovalene. The present invention is, however, not limited to these.

Therefore, as the diamine compound represented by formula (1) is specifically exemplified, it is possible to cite compounds, such as 2-(1-hydroxy-1-trifluoromethyl-2,2,2-trifluoroethyl)-1,8-naphthalene diamine, 4-(1-hydroxy-1-trifluoromethyl-2,2,2-trifluoroethyl)-1,8-naphthalene diamine, 2,7-bis(1-hydroxy-1-trifluoromethyl-2,2,2-trifluoroethyl)-1,8-naphthalene diamine, 2,5-bis(1-hydroxy-1-trifluoromethyl-2,2,2-trifluoroethyl)-1,8-naphthalene diamine, 4,5-bis(1-hydroxy-1-trifluoromethyl-2,2,2-trifluoroethyl)-1,8-naphthalene diamine, 2,4,5-tris(1-hydroxy-1-trifluoromethyl-2,2,2-trifluoroethyl)-1,8-naphthalene diamine, 2,4,7-tris(1-hydroxy-1-trifluoromethyl-2,2,2-trifluoroethyl)-1,8-naphthalene diamine, 2,4,5,7-tetrakis(1-hydroxy-1-trifluoromethyl-2,2,2-trifluoroethyl)-1,8-naphthalene diamine, 2-(1-hydroxy-1-trifluoromethyl-2,2,2-trifluoroethyl)-1,5-naphthalene diamine, 4-(1-hydroxy-1-trifluoromethyl-2,2,2-trifluoroethyl)-1,5-naphthalene diamine, 2,6-bis(1-hydroxy-1-trifluoromethyl-2,2,2-trifluoroethyl)-1,5-naphthalene diamine, 2,8-bis(1-hydroxy-1-trifluoromethyl-2,2,2-trifluoroethyl)-1,5-naphthalene diamine, 4,8-bis(1-hydroxy-1-trifluoromethyl-2,2,2-trifluoroethyl)-1,5-naphthalene diamine, 2,4,6-tris(1-hydroxy-1-trifluoromethyl-2,2,2-trifluoroethyl)-1,5-naphthalene diamine, 2,4,8-tris(1-hydroxy-1-trifluoromethyl-2,2,2-trifluoroethyl)-1,5-naphthalene diamine, 2,4,5,8-tetrakis(1-hydroxy-1-trifluoromethyl-2,2,2-trifluoroethyl)-1,5-naphthalene diamine, 1-(1-hydroxy-1-trifluoromethyl-2,2,2-trifluoroethyl)-2,7-naphthalene diamine, 3-(1-hydroxy-1-trifluoromethyl-2,2,2-trifluoroethyl)-2,7-naphthalene diamine, 1,8-bis(1-hydroxy-1-trifluoromethyl-2,2,2-trifluoroethyl)-2,7-naphthalene diamine, 3,6-bis(1-hydroxy-1-trifluoromethyl-2,2,2-trifluoroethyl)-2,7-naphthalene diamine, 1,6-bis(1-hydroxy-1-trifluoromethyl-2,2,2-trifluoroethyl)-2,7-naphthalene diamine, 1,3,6-tris(1-hydroxy-1-trifluoromethyl-2,2,2-trifluoroethyl)-2,7-naphthalene diamine, 1,3,8-tris(1-hydroxy-1-trifluoromethyl-2,2,2-trifluoroethyl)-2,7-naphthalene diamine, 1,3,6,8-tetrakis(1-hydroxy-1-trifluoromethyl-2,2,2-trifluoroethyl)-2,7-naphthalene diamine, 1-(1-hydroxy-1-trifluoromethyl-2,2,2-trifluoroethyl)-2,6-naphthalene diamine, 3-(1-hydroxy-1-trifluoromethyl-2,2,2-trifluoroethyl)-2,6-naphthalene diamine, 1,5-bis(1-hydroxy-1-trifluoromethyl-2,2,2-trifluoroethyl)-2,6-naphthalene diamine, 1,7-bis(1-hydroxy-1-trifluoromethyl-2,2,2-trifluoroethyl)-2,6-naphthalene diamine, 3,5-bis(1-hydroxy-1-trifluoromethyl-2,2,2-trifluoroethyl)-2,6-naphthalene diamine, 1,3,5-tris(1-hydroxy-1-trifluoromethyl-2,2,2-trifluoroethyl)-2,6-naphthalene diamine, 1,3,7-tris(1-hydroxy-1-trifluoromethyl-2,2,2-trifluoroethyl)-2,6-naphthalene diamine, 1,3,5,7-tetrakis(1-hydroxy-1-trifluoromethyl-2,2,2-trifluoroethyl)-2,6-naphthalene diamine, 2-(1-hydroxy-1-trifluoromethyl-2,2,2-trifluoroethyl)-1,6-naphthalene diamine, 4-(1-hydroxy-1-trifluoromethyl-2,2,2-trifluoroethyl)-1,6-naphthalene diamine, 5-(1-hydroxy-1-trifluoromethyl-2,2,2-trifluoroethyl)-1,6-naphthalene diamine, 7-(1-hydroxy-1-trifluoromethyl-2,2,2-trifluoroethyl)-1,6-naphthalene diamine, 2,5-bis(1-hydroxy-1-trifluoromethyl-2,2,2-trifluoroethyl)-1,6-naphthalene diamine, 2,7-bis(1-hydroxy-1-trifluoromethyl-2,2,2-trifluoroethyl)-1,6-naphthalene diamine, 4,5-bis(1-hydroxy-1-trifluoromethyl-2,2,2-trifluoroethyl)-1,6-naphthalene diamine, 4,7-bis(1-hydroxy-1-trifluoromethyl-2,2,2-trifluoroethyl)-1,6-naphthalene diamine, 2,4,5-tris(1-hydroxy-1-trifluoromethyl-2,2,2-trifluoroethyl)-1,6-naphthalene diamine, 2,4,7-tris(1-hydroxy-1-trifluoromethyl-2,2,2-trifluoroethyl)-1,6-naphthalene diamine, 4,5,7-tris(1-hydroxy-1-trifluoromethyl-2,2,2-trifluoroethyl)-1,6-naphthalene diamine, 2,4,5,7-tetrakis(1-hydroxy-1-trifluoromethyl-2,2,2-trifluoroethyl)-1,6-naphthalene diamine, 2-(1-hydroxy-1-trifluoromethyl-2,2,2-trifluoroethyl)-1,7-naphthalene diamine, 4-(1-hydroxy-1-trifluoromethyl-2,2,2-trifluoroethyl)-1,7-naphthalene diamine, 6-(1-hydroxy-1-trifluoromethyl-2,2,2-trifluoroethyl)-1,7-naphthalene diamine, 8-(1-hydroxy-1-trifluoromethyl-2,2,2-trifluoroethyl)-1,7-naphthalene diamine, 2,6-bis(1-hydroxy-1-trifluoromethyl-2,2,2-trifluoroethyl)-1,7-naphthalene diamine, 2,8-bis(1-hydroxy-1-trifluoromethyl-2,2,2-trifluoroethyl)-1,7-naphthalene diamine, 4,6-bis(1-hydroxy-1-trifluoromethyl-2,2,2-trifluoroethyl)-1,7-naphthalene diamine, 4,8-bis(1-hydroxy-1-trifluoromethyl-2,2,2-trifluoroethyl)-1,7-naphthalene diamine, 2,4,6-tris(1-hydroxy-1-trifluoromethyl-2,2,2-trifluoroethyl)-1,7-naphthalene diamine, 2,4,8-tris(1-hydroxy-1-trifluoromethyl-2,2,2-trifluoroethyl)-1,7-naphthalene diamine, 4,6,8-tris(1-hydroxy-1-trifluoromethyl-2,2,2-trifluoroethyl)-1,7-naphthalene diamine, 2,4,6,8-tetrakis(1-hydroxy-1-trifluoromethyl-2,2,2-trifluoroethyl)-1,7-naphthalene diamine, 2-(1-hydroxy-1-trifluoromethyl-2,2,2-trifluoroethyl)-1,8-anthracene diamine, 4-(1-hydroxy-1-trifluoromethyl-2,2,2-trifluoroethyl)-1,8-anthracene diamine, 2,7-bis(1-hydroxy-1-trifluoromethyl-2,2,2-trifluoroethyl)-1,8-anthracene diamine, 2,5-bis(1-hydroxy-1-trifluoromethyl-2,2,2-trifluoroethyl)-1,8-anthracene diamine, 4,5-bis(1-hydroxy-1-trifluoromethyl-2,2,2-trifluoroethyl)-1,8-anthracene diamine, 2,4,5-tris(1-hydroxy-1-trifluoromethyl-2,2,2-trifluoroethyl)-1,8-anthracene diamine, 2,4,7-tris(1-hydroxy-1-trifluoromethyl-2,2,2-trifluoroethyl)-1,8-anthracene diamine, 2,4,5,7-tetrakis(1-hydroxy-1-trifluoromethyl-2,2,2-trifluoroethyl)-1,8-anthracene diamine, 1-(1-hydroxy-1-trifluoromethyl-2,2,2-trifluoroethyl)-2,7-anthracene diamine, 3-(1-hydroxy-1-trifluoromethyl-2,2,2-trifluoroethyl)-2,7-anthracene diamine, 1,8-bis(1-hydroxy-1-trifluoromethyl-2,2,2-trifluoroethyl)-2,7-anthracene diamine, 3,6-bis(1-hydroxy-1-trifluoromethyl-2,2,2-trifluoroethyl)-2,7-anthracene diamine, 1,6-bis(1-hydroxy-1-trifluoromethyl-2,2,2-trifluoroethyl)-2,7-anthracene diamine, 1,3,6-tris(1-hydroxy-1-trifluoromethyl-2,2,2-trifluoroethyl)-2,7-anthracene diamine, 1,3,8-tris(1-hydroxy-1-trifluoromethyl-2,2,2-trifluoroethyl)-2,7-anthracene diamine, 1,3,6,8-tetrakis(1-hydroxy-1-trifluoromethyl-2,2,2-trifluoroethyl)-2,7-anthracene diamine, 1-(1-hydroxy-1-trifluoromethyl-2,2,2-trifluoroethyl)-2,6-anthracene diamine, 3-(1-hydroxy-1-trifluoromethyl-2,2,2-trifluoroethyl)-2,6-anthracene diamine, 1,5-bis(1-hydroxy-1-trifluoromethyl-2,2,2-trifluoroethyl)-2,6-anthracene diamine, 1,7-bis(1-hydroxy-1-trifluoromethyl-2,2,2-trifluoroethyl)-2,6-anthracene diamine, 3,5-bis(1-hydroxy-1-trifluoromethyl-2,2,2-trifluoroethyl)-2,6-anthracene diamine, 1,3,5-tris(1-hydroxy-1-trifluoromethyl-2,2,2-trifluoroethyl)-2,6-anthracene diamine, 1,3,7-tris(1-hydroxy-1-trifluoromethyl-2,2,2-trifluoroethyl)-2,6-anthracene diamine, 1,3,5,7-tetrakis(1-hydroxy-1-trifluoromethyl-2,2,2-trifluoroethyl)-2,6-anthracene diamine, 2-(1-hydroxy-1-trifluoromethyl-2,2,2-trifluoroethyl)-1,6-anthracene diamine, 4-(1-hydroxy-1-trifluoromethyl-2,2,2-trifluoroethyl)-1,6-anthracene diamine, 5-(1-hydroxy-1-trifluoromethyl-2,2,2-trifluoroethyl)-1,6-anthracene diamine, 7-(1-hydroxy-1-trifluoromethyl-2,2,2-trifluoroethyl)-1,6-anthracene diamine, 2,5-bis(1-hydroxy-1-trifluoromethyl-2,2,2-trifluoroethyl)-1,6-anthracene diamine, 2,7-bis(1-hydroxy-1-trifluoromethyl-2,2,2-trifluoroethyl)-1,6-anthracene diamine, 4,5-bis(1-hydroxy-1-trifluoromethyl-2,2,2-trifluoroethyl)-1,6-anthracene diamine, 4,7-bis(1-hydroxy-1-trifluoromethyl-2,2,2-trifluoroethyl)-1,6-anthracene diamine, 2,4,5-tris(1-hydroxy-1-trifluoromethyl-2,2,2-trifluoroethyl)-1,6-anthracene diamine, 2,4,7-tris(1-hydroxy-1-trifluoromethyl-2,2,2-trifluoroethyl)-1,6-anthracene diamine, 4,5,7-tris(1-hydroxy-1-trifluoromethyl-2,2,2-trifluoroethyl)-1,6-anthracene diamine, 2,4,5,7-tetrakis(1-hydroxy-1-trifluoromethyl-2,2,2-trifluoroethyl)-1,6-anthracene diamine, 2-(1-hydroxy-1-trifluoromethyl-2,2,2-trifluoroethyl)-1,7-anthracene diamine, 4-(1-hydroxy-1-trifluoromethyl-2,2,2-trifluoroethyl)-1,7-anthracene diamine, 6-(1-hydroxy-1-trifluoromethyl-2,2,2-trifluoroethyl)-1,7-anthracene diamine, 8-(1-hydroxy-1-trifluoromethyl-2,2,2-trifluoroethyl)-1,7-anthracene diamine, 2,6-bis(1-hydroxy-1-trifluoromethyl-2,2,2-trifluoroethyl)-1,7-anthracene diamine, 2,8-bis(1-hydroxy-1-trifluoromethyl-2,2,2-trifluoroethyl)-1,7-anthracene diamine, 4,6-bis(1-hydroxy-1-trifluoromethyl-2,2,2-trifluoroethyl)-1,7-anthracene diamine, 4,8-bis(1-hydroxy-1-trifluoromethyl-2,2,2-trifluoroethyl)-1,7-anthracene diamine, 2,4,6-tris(1-hydroxy-1-trifluoromethyl-2,2,2-trifluoroethyl)-1,7-anthracene diamine, 2,4,8-tris(1-hydroxy-1-trifluoromethyl-2,2,2-trifluoroethyl)-1,7-anthracene diamine, 4,6,8-tris(1-hydroxy-1-trifluoromethyl-2,2,2-trifluoroethyl)-1,7-anthracene diamine, 2,4,6,8-tetrakis(1-hydroxy-1-trifluoromethyl-2,2,2-trifluoroethyl)-1,7-anthracene diamine, 2-(1-hydroxy-1-trifluoromethyl-2,2,2-trifluoroethyl)-1,8-phenanthrene diamine, 4-(1-hydroxy-1-trifluoromethyl-2,2,2-trifluoroethyl)-1,8-phenanthrene diamine, 2,7-bis(1-hydroxy-1-trifluoromethyl-2,2,2-trifluoroethyl)-1,8-phenanthrene diamine, 2,5-bis(1-hydroxy-1-trifluoromethyl-2,2,2-trifluoroethyl)-1,8-phenanthrene diamine, 4,5-bis(1-hydroxy-1-trifluoromethyl-2,2,2-trifluoroethyl)-1,8-phenanthrene diamine, 2,4,5-tris(1-hydroxy-1-trifluoromethyl-2,2,2-trifluoroethyl)-1,8-phenanthrene diamine, 2,4,7-tris(1-hydroxy-1-trifluoromethyl-2,2,2-trifluoroethyl)-1,8-phenanthrene diamine, 2,4,5,7-tetrakis(1-hydroxy-1-trifluoromethyl-2,2,2-trifluoroethyl)-1,8-phenanthrene diamine, 1-(1-hydroxy-1-trifluoromethyl-2,2,2-trifluoroethyl)-2,7-phenanthrene diamine, 3-(1-hydroxy-1-trifluoromethyl-2,2,2-trifluoroethyl)-2,7-phenanthrene diamine, 1,8-bis(1-hydroxy-1-trifluoromethyl-2,2,2-trifluoroethyl)-2,7-phenanthrene diamine, 3,6-bis(1-hydroxy-1-trifluoromethyl-2,2,2-trifluoroethyl)-2,7-phenanthrene diamine, 1,6-bis(1-hydroxy-1-trifluoromethyl-2,2,2-trifluoroethyl)-2,7-phenanthrene diamine, 1,3,6-tris(1-hydroxy-1-trifluoromethyl-2,2,2-trifluoroethyl)-2,7-phenanthrene diamine, 1,3,8-tris(1-hydroxy-1-trifluoromethyl-2,2,2-trifluoroethyl)-2,7-phenanthrene diamine, 1,3,6,8-tetrakis(1-hydroxy-1-trifluoromethyl-2,2,2-trifluoroethyl)-2,7-phenanthrene diamine, 1-(1-hydroxy-1-trifluoromethyl-2,2,2-trifluoroethyl)-2,6-phenanthrene diamine, 3-(1-hydroxy-1-trifluoromethyl-2,2,2-trifluoroethyl)-2,6-phenanthrene diamine, 1,5-bis(1-hydroxy-1-trifluoromethyl-2,2,2-trifluoroethyl)-2,6-phenanthrene diamine, 1,7-bis(1-hydroxy-1-trifluoromethyl-2,2,2-trifluoroethyl)-2,6-phenanthrene diamine, 3,5-bis(1-hydroxy-1-trifluoromethyl-2,2,2-trifluoroethyl)-2,6-phenanthrene diamine, 3,7-bis(1-hydroxy-1-trifluoromethyl-2,2,2-trifluoroethyl)-2,6-phenanthrene diamine, 1,3,5-tris(1-hydroxy-1-trifluoromethyl-2,2,2-trifluoroethyl)-2,6-phenanthrene diamine, 1,3,7-tris(1-hydroxy-1-trifluoromethyl-2,2,2-trifluoroethyl)-2,6-phenanthrene diamine, 3,5,7-tris(1-hydroxy-1-trifluoromethyl-2,2,2-trifluoroethyl)-2,6-phenanthrene diamine, 1,3,5,7-tetrakis(1-hydroxy-1-trifluoromethyl-2,2,2-trifluoroethyl)-2,6-phenanthrene diamine, 2-(1-hydroxy-1-trifluoromethyl-2,2,2-trifluoroethyl)-1,6-phenanthrene diamine, 4-(1-hydroxy-1-trifluoromethyl-2,2,2-trifluoroethyl)-1,6-phenanthrene diamine, 5-(1-hydroxy-1-trifluoromethyl-2,2,2-trifluoroethyl)-1,6-phenanthrene diamine, 7-(1-hydroxy-1-trifluoromethyl-2,2,2-trifluoroethyl)-1,6-phenanthrene diamine, 2,5-bis(1-hydroxy-1-trifluoromethyl-2,2,2-trifluoroethyl)-1,6-phenanthrene diamine, 2,7-bis(1-hydroxy-1-trifluoromethyl-2,2,2-trifluoroethyl)-1,6-phenanthrene diamine, 4,5-bis(1-hydroxy-1-trifluoromethyl-2,2,2-trifluoroethyl)-1,6-phenanthrene diamine, 4,7-bis(1-hydroxy-1-trifluoromethyl-2,2,2-trifluoroethyl)-1,6-phenanthrene diamine, 2,4,5-tris(1-hydroxy-1-trifluoromethyl-2,2,2-trifluoroethyl)-1,6-phenanthrene diamine, 2,4,7-tris(1-hydroxy-1-trifluoromethyl-2,2,2-trifluoroethyl)-1,6-phenanthrene diamine, 4,5,7-tris(1-hydroxy-1-trifluoromethyl-2,2,2-trifluoroethyl)-1,6-phenanthrene diamine, 2,4,5,7-tetrakis(1-hydroxy-1-trifluoromethyl-2,2,2-trifluoroethyl)-1,6-phenanthrene diamine, 2-(1-hydroxy-1-trifluoromethyl-2,2,2-trifluoroethyl)-3,6-phenanthrene diamine, 4-(1-hydroxy-1-trifluoromethyl-2,2,2-trifluoroethyl)-3,6-phenanthrene diamine, 2,5-bis(1-hydroxy-1-trifluoromethyl-2,2,2-trifluoroethyl)-3,6-phenanthrene diamine, 2,7-bis(1-hydroxy-1-trifluoromethyl-2,2,2-trifluoroethyl)-3,6-phenanthrene diamine, 4,5-bis(1-hydroxy-1-trifluoromethyl-2,2,2-trifluoroethyl)-3,6-phenanthrene diamine, 2,4,5-tris(1-hydroxy-1-trifluoromethyl-2,2,2-trifluoroethyl)-3,6-phenanthrene diamine, 2,4,7-tris(1-hydroxy-1-trifluoromethyl-2,2,2-trifluoroethyl)-3,6-phenanthrene diamine, 2,4,5,7-tetrakis(1-hydroxy-1-trifluoromethyl-2,2,2-trifluoroethyl)-3,6-phenanthrene diamine, 2,9-bis(1-hydroxy-1-trifluoromethyl-2,2,2-trifluoroethyl)-1,10-phenanthrene diamine, 4,9-bis(1-hydroxy-1-trifluoromethyl-2,2,2-trifluoroethyl)-1,10-phenanthrene diamine, 1,9-bis(1-hydroxy-1-trifluoromethyl-2,2,2-trifluoroethyl)-2,10-phenanthrene diamine, 3,9-bis(1-hydroxy-1-trifluoromethyl-2,2,2-trifluoroethyl)-2,10-phenanthrene diamine, 2,9-bis(1-hydroxy-1-trifluoromethyl-2,2,2-trifluoroethyl)-3,10-phenanthrene diamine, 4,9-bis(1-hydroxy-1-trifluoromethyl-2,2,2-trifluoroethyl)-3,10-phenanthrene diamine, 1,9-bis(1-hydroxy-1-trifluoromethyl-2,2,2-trifluoroethyl)-4,10-phenanthrene diamine, 3,9-bis(1-hydroxy-1-trifluoromethyl-2,2,2-trifluoroethyl)-4,10-phenanthrene diamine, 2,10-bis(1-hydroxy-1-trifluoromethyl-2,2,2-trifluoroethyl)-1,9-phenanthrene diamine, 4,10-bis(1-hydroxy-1-trifluoromethyl-2,2,2-trifluoroethyl)-1,9-phenanthrene diamine, 1,10-bis(1-hydroxy-1-trifluoromethyl-2,2,2-trifluoroethyl)-2,9-phenanthrene diamine, 3,10-bis(1-hydroxy-1-trifluoromethyl-2,2,2-trifluoroethyl)-2,9-phenanthrene diamine, 2,10-bis(1-hydroxy-1-trifluoromethyl-2,2,2-trifluoroethyl)-3,9-phenanthrene diamine, 4,10-bis(1-hydroxy-1-trifluoromethyl-2,2,2-trifluoroethyl)-3,9-phenanthrene diamine, 1,10-bis(1-hydroxy-1-trifluoromethyl-2,2,2-trifluoroethyl)-4,9-phenanthrene diamine, 3,10-bis(1-hydroxy-1-trifluoromethyl-2,2,2-trifluoroethyl)-4,9-phenanthrene diamine, 2,9-bis(1-hydroxy-1-trifluoromethyl-2,2,2-trifluoroethyl)-1,10-naphthacene diamine, 3,8-bis(1-hydroxy-1-trifluoromethyl-2,2,2-trifluoroethyl)-2,9-naphthacene diamine, 1,10-bis(1-hydroxy-1-trifluoromethyl-2,2,2-trifluoroethyl)-2,9-naphthacene diamine, 3,9-bis(1-hydroxy-1-trifluoromethyl-2,2,2-trifluoroethyl)-2,8-naphthacene diamine, 1,7-bis(1-hydroxy-1-trifluoromethyl-2,2,2-trifluoroethyl)-2,8-naphthacene diamine, 2,8-bis(1-hydroxy-1-trifluoromethyl-2,2,2-trifluoroethyl)-1,7-naphthacene diamine, 2,10-bis(1-hydroxy-1-trifluoromethyl-2,2,2-trifluoroethyl)-1,11-pentacene diamine, 3,9-bis(1-hydroxy-1-trifluoromethyl-2,2,2-trifluoroethyl)-2,10-pentacene diamine, 1,11-bis(1-hydroxy-1-trifluoromethyl-2,2,2-trifluoroethyl)-2,9-pentacene diamine, 3,9-bis(1-hydroxy-1-trifluoromethyl-2,2,2-trifluoroethyl)-2,10-pentacene diamine, 1,11-bis(1-hydroxy-1-trifluoromethyl-2,2,2-trifluoroethyl)-2,10-pentacene diamine, and 2,9-bis(1-hydroxy-1-trifluoromethyl-2,2,2-trifluoroethyl)-1,8-pentacene diamine. The present invention is, however, not limited to these.

Herein, there is described a process for synthesizing 2,6-bis(1-hydroxy-1-trifluoromethyl-2,2,2-trifluoroethyl)-1,5-naphthalene diamine represented by formula (7)

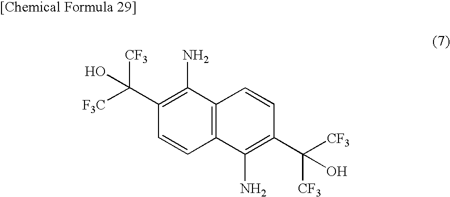

as one of representative examples of the diamine compound represented by formula (1).

This diamine compound is obtained by reacting, for example, 1,5-naphthalene diamine with hexafluoroacetone or hexafluoroacetone trihydrate.

In the case of using hexafluoroacetone, the reaction is conducted by introducing hexafluoroacetone into 1,5-naphthalene diamine as a raw material. Since boiling point of hexafluoroacetone is low (−28° C.), it is preferable to use an apparatus (a cooling apparatus or sealed reactor) for preventing outflow of hexafluoroacetone toward the outside of the reaction system. A sealed reactor is particularly preferable as the apparatus.

In the case of using hexafluoroacetone trihydrate, the reaction can be started by mixing together 1,5-naphthalene diamine as a raw material and hexafluoroacetone trihydrate at the same time. Since boiling point of hexafluoroacetone trihydrate is relatively high (105° C.), its handling is easy as compared with hexafluoroacetone (boiling point: −28° C.). In this case, a sealed container can also be used as the reaction apparatus. It is, however, possible to sufficiently prevent outflow of hexafluoroacetone trihydrate toward the outside of the reaction system, even by allowing tap water (room temperature) to flow through a normal reflux condenser.

The amount of hexafluoroacetone or hexafluoroacetone trihydrate is preferably 2 equivalents to 10 equivalents, more preferably 2.5 equivalents to 5 equivalents, relative to 1,5-naphthalene diamine. The reaction proceeds without problem even by using more than this, but it is not preferable from economical viewpoint.

The present reaction is conducted normally in a temperature range of room temperature to 180° C., preferably 50° C. to 150° C., particularly preferably 90° C. to 27 130° C. A case, in which it is lower than room temperature, is not preferable, since the reaction hardly proceeds. A temperature exceeding 180° C. is not preferable, since side reactions proceed.

Although the present reaction can be conducted without using catalyst, it is possible to accelerate the reaction by using an acid catalyst. As the catalyst used, Lewis acids such as aluminum chloride, iron (III) chloride and boron fluoride, and organic sulfonic acids such as benzenesulfonic acid, camphorsulfonic acid (CSA), methanesulfonic acid, p-toluenesulfonic acid (pTsOH), p-toluenesulfonic acid (pTsOH) monohydrate and pyridinium p-toluene sulfonate (PPTS) are preferable. Of these, aluminum chloride, iron (III) chloride, methanesulfonic acid and p-toluenesulfonic acid (pTsOH) monohydrate are particularly preferable. The amount of the catalyst used is preferably 1 mol % to 50 mol %, particularly preferably 3 mol % to 40 mol %, relative to lmol of 4,4′-oxydianiline. The reaction proceeds without problem even by using more than this, but it is not preferable from economical viewpoint.

Although the present reaction can be conducted without using solvent, it is also possible to use solvent. The solvent to be used is not particularly limited as long as it is not involved in the reaction. An aromatic hydrocarbon, such as xylene, toluene, benzene, anisole, diphenyl ether, nitrobenzene and benzonitrile, or water is preferable. The amount of the solvent to be used is not particularly limited, but use in large amount is not preferable since yield per volume lowers.

In the case of conducting the present reaction in a sealed reactor (autoclave), the mode is different depending on the use of hexafluoroacetone or hexafluoroacetone trihydrate. In the case of using hexafluoroacetone, the reactor is charged firstly with 1,5-naphthalene diamine and according to need catalyst and/or solvent. Then, it is preferable to successively introduce hexafluoroacetone, while increasing the temperature in a manner that the reactor inside pressure does not exceed 0.5 MPa.

In the case of using hexafluoroacetone trihydrate, it is possible to firstly introduce 1,5-naphthalene diamine and a necessary amount of hexafluoroacetone trihydrate. Furthermore, according to need, it is possible to conduct the reaction by introducing catalyst and/or solvent into the reactor.

Although the reaction time of the present reaction is not particularly limited, the optimum reaction time is different depending on the temperature, the amount of catalyst used, etc. Therefore, it is preferable to terminate the present step, after confirming that the raw material has sufficiently been consumed by conducting the reaction, while measuring the progress condition of the reaction by a general-purpose analysis means such as gas chromatography. After the termination of the reaction, it is possible to obtain 2,6-bis(1-hydroxy-1-trifluoromethyl-2,2,2-trifluoroethyl)-1,5-naphthalene diamine by normal means such as extraction, distillation and crystallization. According to need, it is also possible to conduct a purification by column chromatography or recrystallization, etc.

Next, there is described a process for synthesizing 2-(1-hydroxy-1-trifluoromethyl-2,2,2-trifluoroethyl)-1,5-naphthalene diamine, which is a monomer represented by formula (21)

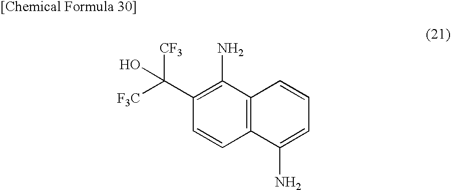

as one of representative examples of formula (1).

The synthesis of this diamine can be conducted in accordance with the above-mentioned synthesis method of 2-(1-hydroxy-1-trifluoromethyl-2,2,2-trifluoroethyl)-1,5-naphthalene diamine, except in that the amount of hexafluoroacetone or hexafluoroacetone trihydrate to be used is reduced.

Specifically, the amount of hexafluoroacetone or hexafluoroacetone trihydrate used upon the synthesis of the present diamine is preferably 1 equivalent to 5 equivalents, more preferably 1.5 equivalents to 3 equivalents, relative to 1,5-naphthalene diamine. The reaction proceeds without problem even by using more than this, but it is not preferable from economical viewpoint.

It is possible to control the number of hexafluoroisopropyl groups to be introduced onto the 1,5-naphthalene skeleton by controlling the amount of hexafluoroacetone or hexafluoroacetone trihydrate used.

The production of other fluorine-containing diamines represented by the formula [1] can also be conducted in accordance with the above-mentioned synthesis method of 2,6-bis(1-hydroxy-1-trifluoromethyl-2,2,2-trifluoroethyl)-1,5-naphthalene diamine.

Next, as an exemplary use of the diamine compound according to the present invention, a process for producing a polymer by polymerizing this diamine compound is explained. This diamine compound is a compound having at least one hexafluoroisopropyl group, and has at least three functional groups at the same time in the molecule. In the case of producing a polymer, these at least three functional groups are effectively used. Specifically, it is preferable to use diamine.

The diamine compound, which is a fluorine-containing polymerizable monomer of the present invention, can be polymerized into the above-mentioned polyamide type polymer compound with good yield by bringing it into contact with a dicarboxylic acid monomer represented by formula (22) or formula (23) to conduct the reaction in a predetermined temperature range. In this case, it is possible to use not only dicarboxylic acid, but also its derivatives, for example, dicarboxylic acid dihalides (halogen is chlorine, bromine, fluorine, or iodine), dicarboxylic acid monoesters, and dicarboxylic diesters.

As the dicarboxylic acid represented by formula (22) or (23) usable in the present invention is exemplified in the form of dicarboxylic acid, it can be exemplified by aliphatic dicarboxylic acids such as oxalic acid, malonic acid, succinic acid, glutaric acid, adipic acid, pimelic acid, suberic acid, azelaic acid, and sebacic acid; and aromatic dicarboxylic acids such as phthalic acid, isophthalic acid, terephthalic acid, 3,3′-dicarboxyldiphenyl ether, 3,4′-dicarboxyldiphenyl ether, 4,4′-dicarboxyldiphenyl ether, 3,3′-dicarboxyldiphenylmethane, 3,4′-dicarboxyldiphenylmethane, 4,4′-dicarboxyldiphenylmethane, 3,3′-dicarboxyldiphenyldifluoromethane, 3,3′-dicarboxyldiphenyldifluoromethane, 3,4′-dicarboxyldiphenyldifluoromethane, 4,4′-dicarboxyldiphenyldifluoromethane, 3,3′-dicarboxyldiphenyl sulfone, 3,4′-dicarboxyldiphenyl sulfone, 4,4′-dicarboxyldiphenyl sulfone, 3,3′-dicarboxyldiphenyl sulfide, 3,4-dicarboxyldiphenyl sulfide, 4,4′-dicarboxyldiphenyl sulfide, 3,3′-dicarboxyldiphenyl ketone, 3,4′-dicarboxyldiphenyl ketone, 4,4′-dicarboxyldiphenyl ketone, 2,2-bis(3-carboxyphenyl)propane, 2,2-bis(3,4′-dicarboxyphenyl)propane, 2,2-bis(4-carboxyphenyl)propane, 2,2-bis(3-carboxyphenyl)hexafluoropropane, 2,2-bis(3,4′-dicarboxyphenyl)hexafluoropropane, 2,2-bis(4-carboxyphenyl)hexafluoropropane, 1,3-bis(3-carboxyphenoxy)benzene, 1,4-bis(3-carboxyphenoxy)benzene, 1,4-bis(4-carboxyphenoxy)benzene, 3,3′-(1,4-phenylenebis(1-methylethylidene))bisbenzoic acid, 3,4′-(1,4-phenylenebis(1-methylethylidene))bisbenzoic acid, 4,4′-(1,4-phenylenebis(1-methylethylidene))bisbenzoic acid, 2,2-bis(4-(3-carboxyphenoxy)phenyl)propane, 2,2-bis(4-(4-carboxyphenoxy)phenyl)propane, 2,2-bis(4-(3-carboxyphenoxy)phenyl)hexafluoropropane, 2,2-bis(4-(4-carboxyphenoxy)phenyl)hexafluoropropane, bis(4-(3-carboxyphenoxy)phenyl)sulfide, bis(4-(4-carboxyphenoxy)phenyl)sulfide, bis(4-(3-carboxyphenoxy)phenyl)sulfone, bis(4-(4-carboxyphenoxy)phenyl)sulfone; perfluorononenyloxy group-containing dicarboxylic acids such as 5-(perfluorononenyloxy)isophthalic acid, 4-(perfluorononenyloxy)phthalic acid, 2-(perfluorononenyloxy)terephthalic acid, and 4-methoxy-5-(perfluorononenyloxy)isophthalic acid; and perfluorohexenyloxy group-containing dicarboxylic acids such as 5-(perfluorohexenyloxy)isophthalic acid, 4-(perfluorohexenyloxy)phthalic acid, 2-(perfluorohexenyloxy)terephthalic acid, and 4-methoxy-5-(perfluorohexenyloxy)isophthalic acid.

As one example of the polymerization reaction, for example, if a fluorine-containing polymerizable monomer represented by the formula [1] of the present invention is reacted with the above dicarboxylic acid monomer (formula [22] or formula [23]), a polymer compound (polyamide resin) represented by the formula [10] is obtained.

This polymerization reaction is not particularly limited in terms of method and condition. For example, it is possible to cite a method in which the above diamine component and an amide-forming derivative of the above dicarboxylic acid are mutually dissolved (melted) at 150° C. or higher to have a reaction without solvent, a method in which the reaction is conducted at high temperature (preferably 150° C. or higher) in an organic solvent, and a method in which the reaction is conducted at a temperature of −20 to 80° C. in an organic solvent.

Usable organic solvent is not particularly limited, as long as both components of the raw materials are dissolved therein. It can be exemplified by amide solvents such as N,N-dimethylformamide, N,N-dimethylacetamide, N-methylformamide, hexamethylphosphoric acid triamide, and N-methyl-2-pyrollidone; aromatic solvents such as benzene, anisole, diphenyl ether, nitrobenzene, and benzonitrile; halogen-series solvents such as chloroform, dichloromethane, 1,2-dichloroethane, and 1,1,2,2-tetrachloroethane; and lactones such as γ-butyrolactone, γ-valerolactone, δ-valerolactone, γ-caprolactone, ε-caprolactone, α-methyl-γ-butyrolactone. It is effective to conduct the reaction with such organic solvent under coexistence with an acid acceptor such as pyridine and triethylamine. In particular, if the above amide solvents are used, these solvents themselves become acid acceptors. Therefore, it is possible to obtain polyamide resins of high degree of polymerization.

It is also possible to turn a diamine compound (a fluorine-containing polymerizable monomer) of the present invention into copolymers by combinations with other diamines and dihydroxyamine, etc. The combinable diamine compound can be exemplified by 3,5-diaminobenzotrifluoride, 2,5-diaminobenzotrifluoride, 3,3′-bistrifluoromethyl-4,4′-diaminobiphenyl, 3,3′-bistrifluoromethyl-5,5′-diaminobiphenyl, bis(trifluoromethyl)-4,4′-diaminodiphenyl, bis(fluorinated alkyl)-4,4′-diaminodiphenyl, dichloro-4,4′-diaminodiphenyl, dibromo-4,4′-diaminodiphenyl, bis(fluorinated alkoxy)-4,4′-diaminodiphenyl, diphenyl-4,4′-diaminodiphenyl, 4,4′-bis(4-aminotetrafluorophenoxy)tetrafluorobenzene, 4,4′-bis(4-aminotetrafluorophenoxy)octafluorobiphenyl, 4,4′-binaphthylamine, o-, m- and p-phenylenediamines, 2,4-diaminotoluene, 2,5-diaminotoluene, 2,4-diaminoxylene, 2,4-diaminodurene, dimethyl-4,4′-diaminodiphenyl, dialkyl-4,4′-diaminodiphenyl, dimethoxy-4,4′-diaminodiphenyl, diethoxy-4,4′-diaminodiphenyl, 4,4′-diaminodiphenylmethane, 4,4′-diaminodiphenyl ether, 3,4′-diaminodiphenyl ether, 4,4′-diaminodiphenylsulfone, 3,3′-diaminodiphenylsulfone, 4,4′-diaminobenzophenone, 3,3′-diaminobenzophenone, 1,3-bis(3-aminophenoxy)benzene, 1,3-bis(4-aminophenoxy)benzene, 1,4-bis(4-aminophenoxy)benzene, 4,4′-bis(4-aminophenoxy)biphenyl, bis(4-(3-aminophenoxy)phenyl)sulfone, bis(4-(4-aminophenoxy)phenyl)sulfone, 2,2-bis(4-(4-aminophenoxy)phenyl)propane, 2,2-bis(4-(4-aminophenoxy)phenyl)hexafluoropropane, 2,2-bis(4-(3-aminophenoxy)phenyl)propane, 2,2-bis(4-(3-aminophenoxy)phenyl)hexafluoropropane, 2,2-bis(4-(4-amino-2-trifluoromethylphenoxy)phenyl)hexafluoropropane, 2,2-bis(4-(3-amino-5-trifluoromethylphenoxy)phenyl)hexafluoropropane, 2,2-bis(4-aminophenyl)hexafluoropropane, 2,2-bis(3-aminophenyl)hexafluoropropane, 2,2-bis(3-amino-4-hydroxyphenyl)hexafluoropropane, 2,2-bis(3-amino-4-methylphenyl)hexafluoropropane, 4,4′-bis(4-aminophenoxy)octafluorobiphenyl, 4,4′-diaminobenzanilide, etc. At least two of these can be combined. In this case, it becomes a copolymerization composition modified with polybenzoxazole.

It is also possible to use a diamine compound of the present invention by protecting the hexafluoropropyl group to introduce a protecting group (acid-labile group) that is released by acid. Acid-labile groups can be used without limitation, as long as they are groups that generate release by the effect of photoacid generator, hydrolysis, etc. As specific examples, it is possible to cite alkykoxycarbonyl groups such as tert-butoxycarbonyl group, tert-amyloxycarbonyl group, methoxycarbonyl group, and ethoxycarbonyl group; acetal groups such as methoxymethyl group, ethoxyethyl group, butoxyethyl group, cyclohexyloxyethyl group, and benzyloxyethyl group; silyl groups of trimethylsilyl group, ethyldimethylsilyl group, methyldiethylsilyl group, and triethylsilyl group; acyl groups such as acetyl group, propionyl group, butyryl group, heptanoyl group, hexanoyl group, valeryl group, and pivaloyl group.

By introducing an acid-releasing group (acid-labile group), a polymer compound obtained by polymerization using a fluorine-containing polymerizing diamine monomer of the present invention can be used as a resist material. That is, the hexafluoroisopropanol group in the molecule is protected with an acid-labile protecting group. Then, it is mixed with a photoacid generator to produce a resist. By exposing this, the acid labile group is released, thereby forming a hexafluoroisopropanol group. As a result, an alkali development becomes possible. Thus, it is useful as a positive-type resist or photosensitive material.

A fluorine-containing polymerizing monomer of the present invention can be used with other functional groups. For example, it is possible to introduce a crosslinking site by providing an unsaturated bond. For example, if a fluorine-containing polymerizing monomer of the present invention is reacted with maleic anhydride, it turns into a bismaleimide. With this, it is possible to introduce a double bond. This compound is useful as a crosslinking agent.

As a partner monomer of the diamine compound, which is a fluorine-containing polymerizable monomer of the present invention, it is also possible to use a tetracarboxylic acid derivative, for example, a tetracarboxylic acid dianhydride monomer represented by the formula [24]. The tetracarboxylic acid dianhydride monomer of this case can be used without particular limitation, as long as it has a structure generally used as a polyamide acid or polyimide raw material.

Such tetracarboxylic acid dianhydride is not particularly limited in structure. For example, it is possible to cite benzenetetracarboxylic acid dianhydride (pyromellitic acid dianhydride; PMDA), trifluoromethylbenzenetetracarboxylic acid dianhydride, bistrifluoromethylbenzenetetracarboxylic acid dianhydride, difluorobenzenetetracarboxylic acid dianhydride, naphthalenetetracarboxylic acid dianhydride, biphenyltetracarboxylic acid dianhydride, terphenyltetracarboxylic acid dianhydride, hexafluoroisopropylidenediphthalic acid dianhydride, oxydiphthalic acid dianhydride, bicyclo(2,2,2)oct-7-ene-2,3,5,6-tetracarboxylic acid dianhydride, 2,2-bis(3,4-dicarboxyphenyl)hexafluoropropanoic acid dianhydride (6FDA), 2,3,4,5-thiophenetetracarboxylic acid dianhydride, 2,5,6,2′,5′,6′-hexafluoro-3,3′,4,4′-biphenyltetracarboxylic acid dianhydride, bis(3,4-dicarboxyphenyl)sulfonic acid dianhydride, 3,4,9,10-perylenetetracarboxylic acid dianhydride, etc. In particular, pyromellitic acid and 6FDA are preferable. These tetracarboxylic acid dianhydrides may be used alone or in a mixture of at least two. In the present invention, in connection with the ratio in use of the tetracarboxylic acid dianhydride and the amine component, it is used in 0.9-1.1 moles, preferably 0.95-1.05 moles, more preferably 0.98-1.03 moles, relative to 1 mole of the tetracarboxylic acid dianhydride. If it is outside of this range, the molar ratio balance is lost, and its properties are impaired. Therefore, it is not preferable.

Regarding method and condition of the polymerization reaction, it is possible to use a polymerization method and a polymerization condition, which are similar to those of the reactions with dicarboxylic acids. The usable solvent is not particularly limited, as long as both components of the raw materials are dissolved therein. It is possible to use a solvent that is similar to those in the reactions with the dicarboxylic acids. It can be exemplified by amide solvents such as N,N-dimethylformamide, N,N-dimethylacetamide, N-methylformamide, hexamethylphosphoric triamide, and N-methyl-2-pyrolidone; aromatic solvents such as benzene, anisole, diphenyl ether, nitrobenzene, and benzonitrile; halogen-series solvents such as chloroform, dichloromethane, 1,2-dichloroethane, and 1,1,2,2-tetrachloroethane; lactones such as γ-butyrolactone, γ-valerolactone, δ-valerolactone, γ-caprolactone, ε-caprolactone, and α-methyl-γ-butyrolactone. It is effective to conduct the reaction with such organic solvent under coexistence with an acid acceptor such as pyridine and triethylamine.

Similar to the reactions with dicarboxylic acids, it can also be turned into polyimide copolymers in the case of a combination with other diamines and dihydroxyamines. As a combinable diamine compound, it is possible to use the above diamine. Similar to the above, it can also be a combination of at least two.

It is possible to convert polyamide type polymer compounds obtained by the above-mentioned process into hetero ring type polymer compounds represented by formulas (11), (13), (15), (16), (18), etc.

There is no particular limitation in the conditions of the dehydration ring-closure reaction. The cyclization can be conducted by various methods that accelerate the dehydration condition, such as heat and acid catalyst.

In the case of cyclization (dehydration and ring closure), it is possible to conduct a resin modification accompanied with significant changes in terms of physical properties, such as heat resistance improvement, dissolution change, lowering in refractive index and dielectric constant, and achievement of water repellency and oil repellency. In particular, a hetero ring type polymer compound of the present invention is further improved in heat resistance, since it has a cyclic structure in the molecule.

Such excellent physical properties are derived from the basic skeleton of the hetero ring type polymer compounds represented by the formulas (11), (13), (15), (16), (18), etc. Therefore, it is preferable that the number of these repeating units is higher relative to the number of repeating units of the whole of the polymer compound. It is preferably 80% or greater, more preferably 90% or greater. The polymer compound group shown in Examples, in which these units occupy 100%, is a particularly preferable embodiment.

The fluorine-containing polymer of the present invention can be used in the condition of a varnish dissolved in an organic solvent or in the powder condition, film condition, or solid condition. Upon this, according to need, the obtained polymer may be mixed with an additive such as oxidation stabilizer, filler, silane coupling agent, photosensitive agent, photopolymerization initiator and sensitizer. In the case of using it as a varnish, it can be applied to a substrate, such as glass, silicon wafer, metal, metal oxide, ceramic, and resin, by a method normally used, such as spin coating, spraying, flow coating, impregnation coating, and brush coating.

### Examples

Next, the present invention is described in more detail by examples.

### Example 1

### Production of 2,6-bis(1-hydroxy-1-trifluoromethyl-2,2,2-trifluoroethyl)-1,5-naphthalene diamine

A 100 ml sealed container (autoclave) made of glass was charged with 4.05 g (25.6 mmol) of 1,5-naphthalene diamine, 0.49 g (2.6 mmol) of p-toluenesulfonic acid monohydrate, and 45.6 g (207.4 mmol, 8,1 equivalents) of hexafluoroacetone trihydrate, and the inside of the system was turned into under nitrogen atmosphere. Then, the temperature increase was started. After the reaction was conducted at an inside temperature of 120° C. for 46 hours, the reaction liquid was cooled down.

The reaction liquid was found by gas chromatography (GC) analysis to be 93% of the target compound, 2,6-bis(1-hydroxy-1-trifluoromethyl-2,2,2-trifluoroethyl)-1,5-naphthalene diamine and 7% of 2-(1-hydroxy-1-trifluoromethyl-2,2,2-trifluoroethyl)-1,5-naphthalene diamine. After adding 25 mL of water to the reaction liquid, stirring was conducted. After filtering this mixed liquid, the recovered solid was dissolved in methanol. After activated carbon treatment, it was separated by filtration with celite. The obtained methanol solution was subjected to crystallization in water, and the obtained crystals were dried under reduced pressure. 10.20 g (yield 81%, purity 99%) of 2,6-bis(1-hydroxy-1-trifluoromethyl-2,2,2-trifluoroethyl)-1,5-naphthalene diamine, the target compound, was obtained.

[Physical properties of 2,6-bis(1-hydroxy-1-trifluoromethyl-2,2,2-trifluoroethyl)-1,5-naphthalene diamine] A pale-purple color solid at ordinary temperature. ¹H-NMR (standard substance: TMS, solvent: DMSO-D₆) σ (ppm): 6.27 (s, 4H), 7.20 (d, 2H, J=9.2 Hz), 7.43 (d, 2H, J=9.2 Hz), 9.87 (s, 2H). ¹⁹F-NMR (standard substance: CCl₃F, solvent: DMSO-D₆) σ (ppm): −72.23 (s, 12F).

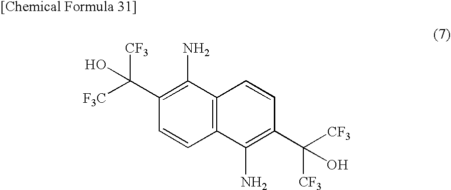

### Example 2

Production of 2,7-bis(1-hydroxy-1-trifluoromethyl-2,2,2-trifluoroethyl)-1,6-anthracene diamine 5.53 g (yield 40%, purity 99%) of 2,7-bis(1-hydroxy-1-trifluoromethyl-2,2,2-trifluoroethyl)-1,6-anthracene diamine, the target compound, was obtained by a method similar to that of Example 1 from 5.33 g (25.6 mmol) of 1,6-anthracene diamine, 0.49 g (2.6 mmol) of p-toluenesulfonic acid monohydrate, and 45.6 g (207.4 mmol, 8.1 equivalents) of hexafluoroacetone trihydrate.

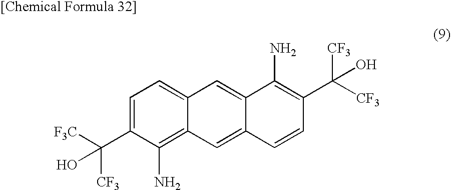

### Example 3

### Synthesis of model compound [2,6-bis(1-hydroxy-1-trifluoromethyl-2,2,2-trifluoroethyl)-1,5-bis(benzoylamino)naphthalene]

A 200 ml, three-necked, round-bottom flask was charged with 2.00 g (4.1 mmol) of 2,6-bis(1-hydroxy-1-trifluoromethyl-2,2,2-trifluoroethyl)-1,5-naphthalene diamine, 1.29 g (16.3 mmol, 4 equivalents) of pyridine, and 30 mL of tetrahydrofuran, and the inside of the system was turned into nitrogen atmosphere. Under room temperature, 1.06 g (8.6 mmol, 2.1 equivalents) of benzoyl chloride was added in dropwise manner. Stirring was conducted at room temperature for 2 hours, and stirring was conducted at 60 degrees for 24 hours. After the reaction, the reaction liquid was introduced into water, and the obtained solid was separated by filtration. The recovered solid was dissolved in methanol, and this was subjected to crystallization in water, and the obtained crystals were dried under reduced pressure. 0.80 g (yield 29%, purity 99%) of 2,6-bis(1-hydroxy-1-trifluoromethyl-2,2,2-trifluoroethyl)-1,5-bis(benzoylamino)naphthalene, the target compound, was obtained.

[Physical properties of 2,6-bis(1-hydroxy-1-trifluoromethyl-2,2,2-trifluoroethyl)-1,5-bis(benzoylamino)naphthalene] A white color solid at ordinary temperature. ¹H-NMR (standard substance: TMS, solvent: DMSO-D₆) σ (ppm): 7.54-7.68 (m, 6H), 7.84 (d, 2H, J=9.2 Hz), 7.96 (d, 2H, J=9.2 Hz), 8.06-8.12 (m, 4H), 9.19 (s, 2H), 10.08 (s, 2H). ¹⁹F-NMR (standard substance: CCl₃F, solvent: DMSO-D₆) σ (ppm): −71.42 (s, 12F).

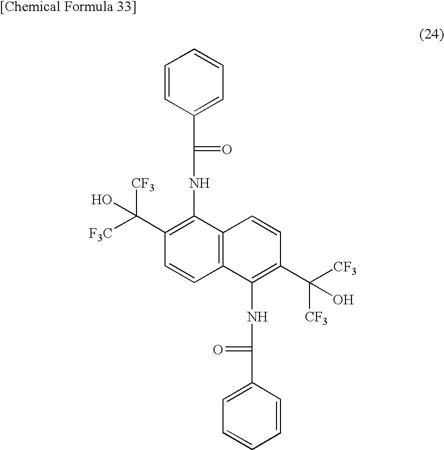

By subjecting the compound represented by formula (24) to a heating treatment at 400° C. for 30 minutes, a compound represented by formula (25) after dehydration and ring closure was obtained.

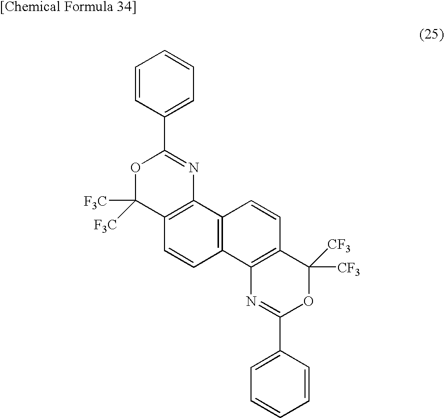

### Example 4

Using 2,6-bis(1-hydroxy-1-trifluoromethyl-2,2,2-trifluoroethyl)-1,5-naphthalene diamine synthesized in Example 1, a polymerization reaction was conducted with isophthalic chloride.

In the polymerization, a sufficiently dried, sealed, 100 ml, three-necked flask equipped with a stirrer and made of glass was charged with 40 g of dimethylacetamide and 0.01 mol (4.90 g) of 2,6-bis(1-hydroxy-1-trifluoromethyl-2,2, 2-trifluoroethyl)-1,5-naphthalene diamine. To become homogeneous, stirring was conducted while bubbling nitrogen. Therein 0.01 mol (2.02 g) of isophthalic chloride was added, and the polymerization was allowed to proceed while stirring for 5 hours. Then, it was reprecipitated in a large amount of methanol, followed by filtration and then drying under reduced pressure, thereby obtaining 5.90 g (yield 95%) of a polymer represented by formula (26). Intrinsic viscosity of the polymer was 1.20 dL/g.

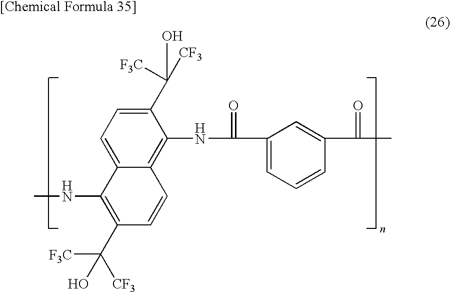

### Example 5

5 g of the polymer represented by formula (26) was dissolved in 20 g of γ-butyrolactone. The γ-butyrolactone solution was developed onto a glass substrate and dried at 120° C., 200° C. and 300° C. for 2 hours respectively, thereby obtaining a transparent film. From IR measurement, structure of the obtained film was found to be a polymer represented by formula (27).

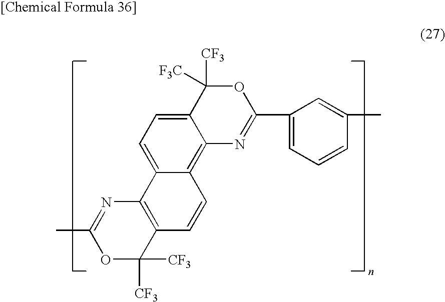

Glass transition temperature of the film was 260° C. from DSC measurement, and thermal expansion coefficient thereof was 50 ppm/K from TMA measurement. As compared with Comparative Example 1 and Comparative Example 2, it was found to be improved such that glass transition temperature was higher and thermal expansion coefficient was lower. It is assumed that this was caused by introducing a rigid naphthalene structure into the diamine side.

### Example 6

A polymer film represented by formula (28) was obtained by a method similar to those of Examples 4 and 5 by using 2,7-bis(1-hydroxy-1-trifluoromethyl-2,2,2-trifluoroethyl)-1,6-anthracene diamine, synthesized by Example 2, in place of 2,6-bis(1-hydroxy-1-trifluoromethyl-2,2,2-trifluoroethyl)-1,5-naphthalene diamine, and by using 4,4′-biphenyldicarboxylic acid chloride in place of isophthalic chloride The obtained film was 270° C. in glass transition temperature and 40 ppm/K in thermal expansion coefficient. As compared with Comparative Example 1 and Comparative Example 2, it was found to be improved such that glass transition temperature was higher and thermal expansion coefficient was lower. It is assumed that this was caused by introducing a rigid anthracene structure into the diamine side.

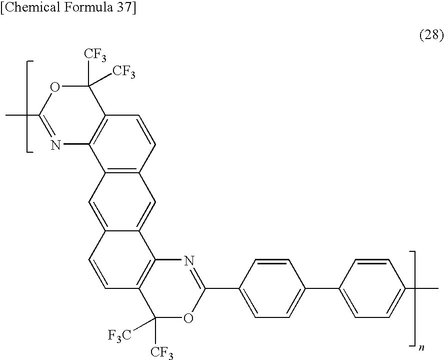

### Comparative Example 1

A polymer film represented by formula (29) was obtained by a method similar to those of Examples 6 and 7 by using 3,3′-bis(1-hydroxy-1-trifluoromethyl-2,2,2-trifluoroethyl)-4,4′-oxydianiline in place of 2,6-bis(1-hydroxy-1-trifluoromethyl-2,2,2-trifluoroethyl)-1,5-naphthalene diamine. The obtained film was 190° C. in glass transition temperature and 90 ppm/K in thermal expansion coefficient.

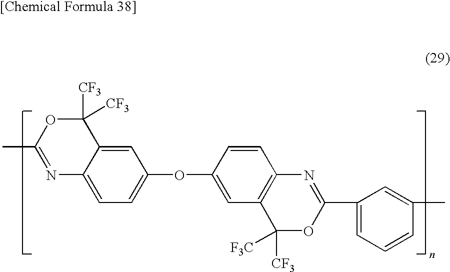

### Comparative Example 2

A polymer film represented by formula (30) was obtained by a method similar to those of Examples 4 and 5 by using 4,4′-biphenyldicarboxylic acid chloride in place of isophthalic chloride. The obtained film was 220° C. in glass transition temperature and 85 ppm/K in thermal expansion coefficient. As compared with Comparative Example 1, it was found that glass transition temperature became higher, but thermal expansion coefficient had almost no change.

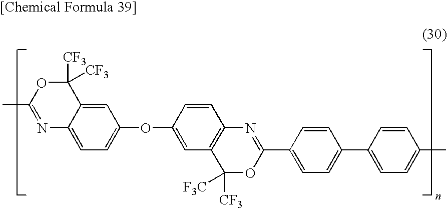

1. A fluorine-containing diamine represented by formula (1) 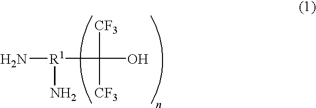
wherein R¹ represents a condensed polycyclic aromatic hydrocarbon group, and the condensed polycyclic aromatic hydrocarbon group may contain N atom, O atom or S atom as a hetero atom and may have a functional group containing an N atom, O atom or S atom as a substituent n represents an integer of 1 or greater and at least one —C(CF₃)₂OH group and at least one —NH₂ group are in a relation such that they are attached to adjacent carbons of carbon atoms constituting the condensed polycyclic aromatic hydrocarbon group.

2. A fluorine-containing diamine according to claim 1, which is represented by formula (2) 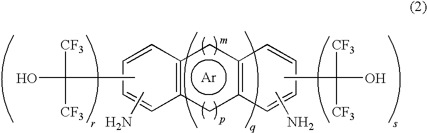
wherein each of m and p is independently an integer of 0, 1 or 2, and m+p≦2. q is 0 or an integer of 1 or greater;
each of r and s is independently an integer of 0-3, and (r+s) is 1 or greater; at least one —C(CF3)₂OH group and at least one —NH₂ group are in a relation such that they are attached to adjacent carbons of carbon atoms constituting the condensed polycyclic aromatic hydrocarbon group;
in the formula (2), a part represented by the following formula 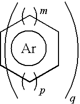
represents a monocyclic aromatic ring or condensed polycyclic type aromatic ring, may contain N atom, O atom or S atom as a hetero atom, and may have a functional group containing an N atom, O atom or S atom as a substituent.

3. A fluorine-containing diamine according to claim 2, which is represented by formula (3) 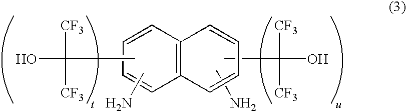
wherein each of t and u is independently an integer of 0-3, and (t+u) is 1 or greater; and at least one —C(CF₃)₂OH group and at least one —NH₂ group are in a relation such that they are attached to adjacent carbons of carbon atoms constituting the condensed polycyclic aromatic hydrocarbon group.

4. A fluorine-containing diamine according to claim 2, which is represented by formula (4) 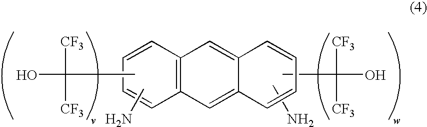
wherein each of v and w is independently an integer of 0-3, and (v+w) is 1 or greater; and at least one —C(CF3)₂OH group and at least one —NH₂ group are in a relation such that they are attached to adjacent carbons of carbon atoms constituting the condensed polycyclic aromatic hydrocarbon group.

5. A fluorine-containing diamine according to claim 2, which is represented by formula (5) 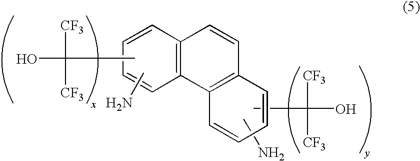
wherein each of x and y is independently an integer of 0-3, and (x+y) is 1 or greater; and at least one —C(CF₃)₂OH group and at least one —NH₂ group are in a relation such that they are attached to adjacent carbons of carbon atoms constituting the condensed polycyclic aromatic hydrocarbon group.

6. A fluorine-containing diamine according to claim 1, which is represented by formula (6) 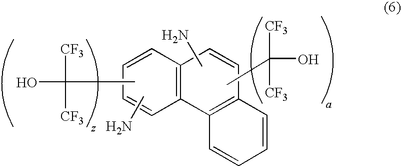
wherein a is 0 or 1, z is an integer of 0-3, and (a+z) is 1 or greater; and at least one —C(CF3)₂OH group and at least one —NH₂ group are in a relation such that they are attached to adjacent carbons of carbon atoms constituting the condensed polycyclic aromatic hydrocarbon group.

7. A fluorine-containing diamine according to claim 3, which is represented by formula (7)

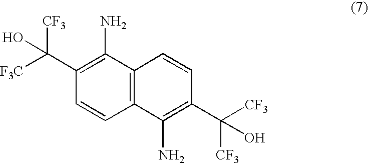

8. A fluorine-containing diamine according to claim 3, which is represented by formula (8)

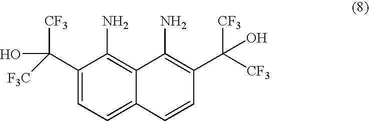

9. A fluorine-containing diamine according to claim 4, which is represented by formula (9)

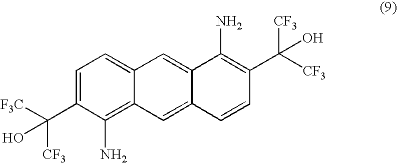

10. A polymer compound obtained by bringing a fluorine-containing diamine according to claim 1 into contact with a dicarboxylic acid derivative represented by formula (22) or (23) 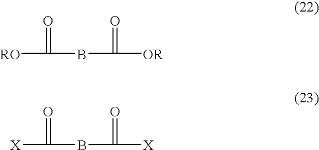
or a tetracarboxylic acid dianhydride monomer represented by formula (24) 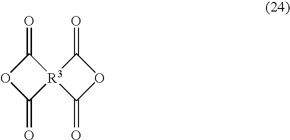
thereby conducting a reaction.

11. A polymer compound obtained by subjecting a polymer compound according to claim 10 to dehydration and ring closure.

12. A polymer compound containing at least a repeating unit represented by the following formula (10) 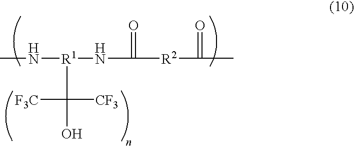
wherein R¹ represents a condensed polycyclic aromatic hydrocarbon group, and the condensed polycyclic aromatic hydrocarbon group may contain N atom, O atom or S atom as a hetero atom and may have a functional group containing an N atom, O atom or S atom as a substituent;
n represents an integer of 1 or greater;
R² is a bivalent organic group containing at least one member selected from the group consisting of aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

13. A polymer compound containing at least a repeating unit represented by the following formula (11) 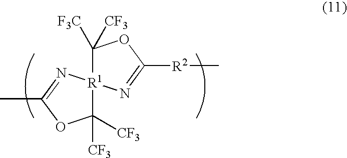
wherein R¹ represents a condensed polycyclic aromatic hydrocarbon group, and the condensed polycyclic aromatic hydrocarbon group may contain N atom, O atom or S atom as a hetero atom and may have a functional group containing an N atom, O atom or S atom as a substituent;
R² is a bivalent organic group containing at least one member selected from the group consisting of aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

14. A polymer compound according to claim 12, wherein the repeating unit of claim 12 is a repeating unit represented by the following formula (12) 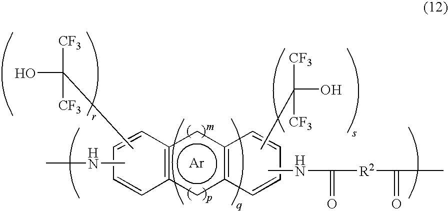
wherein each of m and p is independently an integer of 0, 1 or 2, and m+p≦2;
q is 0 or an integer of 1 or greater;
each of r and s is independently an integer of 0-3, and (r+s) is 1 or greater;
at least one —C(CF3)₂OH group and at least one —NH₂ group are in a relation such that they are attached to adjacent carbons of carbon atoms constituting the condensed polycyclic aromatic hydrocarbon group;
R² is a bivalent organic group containing at least one member selected from the group consisting of aliphatic rings, aromatic rings and alkylene groups, and may contain fluorine, chlorine, oxygen, sulfur or nitrogen, and hydrogens of R² may partly be replaced with an alkyl group, fluoroalkyl group, carboxyl group, hydroxy group or cyano group; and
in the formula (12), a part represented by the following formula 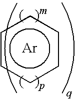
represents a monocyclic aromatic ring or condensed polycyclic aromatic ring, may contain N atom, O atom or S atom as a hetero atom, and may have a functional group containing an N atom, O atom or S atom as a substituent.

15. A polymer compound according to claim 13, wherein the repeating unit of claim 13 is a repeating unit represented by the following formula (13) 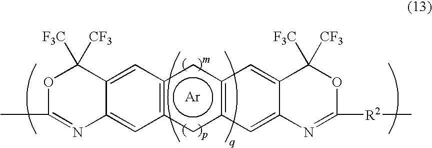
wherein each of m and p is independently an integer of 0, 1 or 2, and m+p≦2;
q is 0 or an integer of 1 or greater;
R² is a bivalent organic group containing at least one member selected from the group consisting of aliphatic rings, aromatic rings and alkylene groups, and may contain fluorine, chlorine, oxygen, sulfur or nitrogen, and hydrogens of R² may partly be replaced with an alkyl group, fluoroalkyl group, carboxyl group, hydroxy group or cyano group; and
in the formula (13), a part represented by the following formula 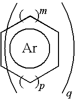
represents a monocyclic aromatic ring or condensed polycyclic aromatic ring, may contain an N atom, O atom or S atom as a hetero atom, and may have a functional group containing an N atom, O atom or S atom as a substituent.

16. A polymer compound according to claim 14, wherein the repeating unit of claim 14 is a repeating unit represented by the following formula (14) 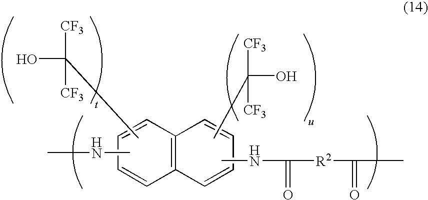
wherein each of t and u is independently an integer of 0-3, and (t+u) is 1 or greater; at least one —C(CF₃)₂OH group and at least one —NH₂ group are in a relation such that they are attached to adjacent carbons of carbon atoms constituting the condensed polycyclic aromatic hydrocarbon group; R² is a bivalent organic group containing at least one member selected from the group consisting of aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

17. A polymer compound according to claim 15, wherein the repeating unit of claim 15 is a repeating unit represented by the following formula (15) 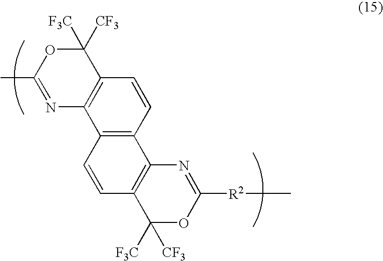
wherein R² is a bivalent organic group containing at least one member selected from the group consisting of aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

18. A polymer compound wherein the repeating unit of claim 15 is a repeating unit represented by the following formula (16) 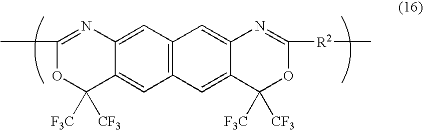
wherein R² is a bivalent organic group containing at least one member selected from the group consisting of aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

19. A polymer compound according to claim 14, wherein the repeating unit of claim 14 is a repeating unit represented by the following formula (17) 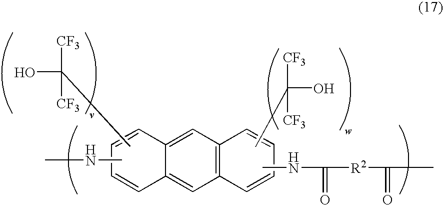
wherein each of v and w is independently an integer of 0-3, and (v+w) is 1 or greater; at least one —C(CF₃)₂OH group and at least one —NH₂ group are in a relation such that they are attached to adjacent carbons of carbon atoms constituting the condensed polycyclic aromatic hydrocarbon group; R² is a bivalent organic group containing at least one member selected from aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

20. A polymer compound according to claim 15, wherein the repeating unit of claim 15 is a repeating unit represented by the following formula (18) 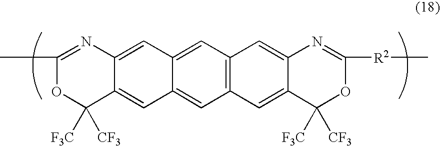
wherein R² is a bivalent organic group containing at least one member selected from the group consisting of aliphatic rings, aromatic rings and alkylene groups, and may contain oxygen, sulfur or nitrogen as a hetero atom, and hydrogens of R² may partly be replaced with an alkyl group, fluorine, chlorine, fluoroalkyl group, carboxyl group, hydroxy group or cyano group.

21. A process for producing a polymer compound according to claim 13, comprising:
(a) bringing a diamine into contact with a dicarboxylic acid compound represented by formula (22) or (23) 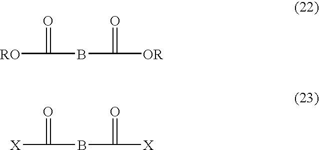
or a tetracarboxylic acid dianhydride monomer represented by formula (24) 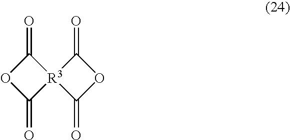
thereby conducting a reaction to produce a polymer compound according to claim 12, followed by subjecting the polymer compound to dehydration and ring closure;
wherein in the formulas (22) and (23),
each R is independently a group selected from hydrogen, C₁₋₁₀ alkyl groups and benzyl group,
B is a bivalent organic group containing at least one member selected from the group consisting of aliphatic rings, aromatic rings and alkylene groups, and may contain oxygen, sulfur or nitrogen, and hydrogens of B may partly be replaced with an alkyl group, fluorine, chlorine, fluoroalkyl group, carboxyl group, hydroxy group or cyano group; and
X represents a halogen atom and wherein in the formula (24), R³ is a tetravalent organic group containing at least one member selected from the group consisting of aliphatic rings, aromatic rings and alkylene groups, and may contain fluorine, chlorine, oxygen, sulfur, or nitrogen, and hydrogens of R³ may partly be replaced with an alkyl group, fluoroalkyl group, carboxyl group, hydroxy group or cyano group.
